(19) United States
(12) Patent Application Publication
(10) Pub. No.: US 2026/0165719 A1
(43) Pub. Date: Jun. 18, 2026

(54) ASPIRATION CATHETERS HAVING ADJUSTABLE FLAPS

(71) Applicant: Covidien LP, Mansfield, MA (US)

(72) Inventors: Danyong Zeng, Aliso Viejo, CA (US); Tejashri Kumar, Fountain Valley, CA (US); Omarinda S. Nichols, Lakewood, CA (US)

(21) Appl. No.: 19/419,254

(22) Filed: Dec. 15, 2025

Related U.S. Application Data
(60) Provisional application No. 63/734,641, filed on Dec. 16, 2024.

(51) Int. Cl. A61B 17/22; A61B 17/00; A61B 90/00; A61M 25/00

(52) U.S. Cl. CPC A61B 17/22; A61B 90/03; A61M 25/007; A61B 2017/00544; A61B 2017/00561; A61B 2017/00778; A61B 2017/22079; A61B 2090/033; A61B 2217/005; A61M 2025/0078; A61M 2205/3344; A61M 2210/12

(57) ABSTRACT

Thrombectomy devices and associated methods and systems are disclosed herein. According to some embodiments, the present technology includes a thrombectomy device including a tubular member having a proximal end region configured to be disposed extracorporeally and a distal end region configured to be disposed at an intravascular treatment site. The tubular member can include a sidewall defining a lumen extending from the proximal end region to the distal end region, and a plurality of side openings in the sidewall in the distal end region. The thrombectomy device can further include at least one adjustable flap configured to at least partially cover a side opening of the plurality of side openings of the tubular member. The adjustable flap may have a first end coupled to the tubular member and a free second end.

### CROSS-REFERENCE TO RELATED APPLICATIONS

This application claims the benefit of and priority to U.S. Provisional Patent Application Ser. No. 63/734,641 filed Dec. 16, 2024, the entire disclosure of which is incorporated by reference herein.

### TECHNICAL FIELD

The present technology relates to systems and methods for removing obstructions from body lumens. Some embodiments of the present technology relate to aspiration catheters having adjustable flaps.

### BACKGROUND

Many medical procedures use medical device(s) to remove an obstruction (such as clotting material) from a body lumen, vessel, or other organ. An inherent risk in such procedures is that mobilizing or otherwise disturbing the obstruction can potentially create further harm if the obstruction or a fragment thereof dislodges from the retrieval device. If all or a portion of the obstruction breaks free from the device and flows downstream, it is highly likely that the free material will become trapped in smaller and more tortuous anatomy. In many cases, the physician will no longer be able to use the same retrieval device to again remove the obstruction because the device may be too large and/or immobile to move the device to the site of the new obstruction.

Procedures for treating ischemic stroke by restoring flow within the cerebral vasculature are subject to the above concerns. The brain relies on its arteries and veins to supply oxygenated blood from the heart and lungs and to remove carbon dioxide and cellular waste from brain tissue. Blockages that interfere with this blood supply eventually cause the brain tissue to stop functioning. If the disruption in blood occurs for a sufficient amount of time, the continued lack of nutrients and oxygen causes irreversible cell death. Accordingly, it is desirable to provide immediate medical treatment of an ischemic stroke.

To access the cerebral vasculature, a physician typically advances a catheter from a remote part of the body (typically a leg) through the abdominal vasculature and into the cerebral region of the vasculature. Once within the cerebral vasculature, the physician deploys a device for retrieval of the obstruction causing the blockage, for example an aspiration catheter. Concerns about dislodged obstructions or the migration of dislodged fragments increases the duration of the procedure at a time when restoration of blood flow is paramount. Furthermore, a physician might be unaware of one or more fragments that dislodge from the initial obstruction and cause blockage of smaller more distal vessels. Accordingly, there remains a need for improved devices and methods that can remove occlusions from body lumens and/or vessels.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 illustrates a side view of an example aspiration catheter that can be improved using the systems and methods described herein.

FIG. 2 illustrates a perspective view of a thrombectomy system, in accordance with embodiments of the present technology.

FIG. 3 illustrates a side view of an example distal end region of a thrombectomy device of the thrombectomy system of FIG. 2, in accordance with embodiments of the present technology.

FIG. 4 illustrates a side view of an example distal end region of a thrombectomy device of the thrombectomy system of FIG. 2, in accordance with embodiments of the present technology.

FIG. 5 illustrates a side view of an example distal end region of a thrombectomy device of the thrombectomy system of FIG. 2, in accordance with embodiments of the present technology.

FIG. 6 illustrates a side view of an example distal end region of a thrombectomy device of the thrombectomy system of FIG. 2, in accordance with embodiments of the present technology.

FIGS. 7A-7C illustrate a method of thrombectomy within a blood vessel, in accordance with embodiments of the present technology.

FIGS. 8A-8C illustrate a method of thrombectomy within a blood vessel, in accordance with embodiments of the present technology.

### DETAILED DESCRIPTION

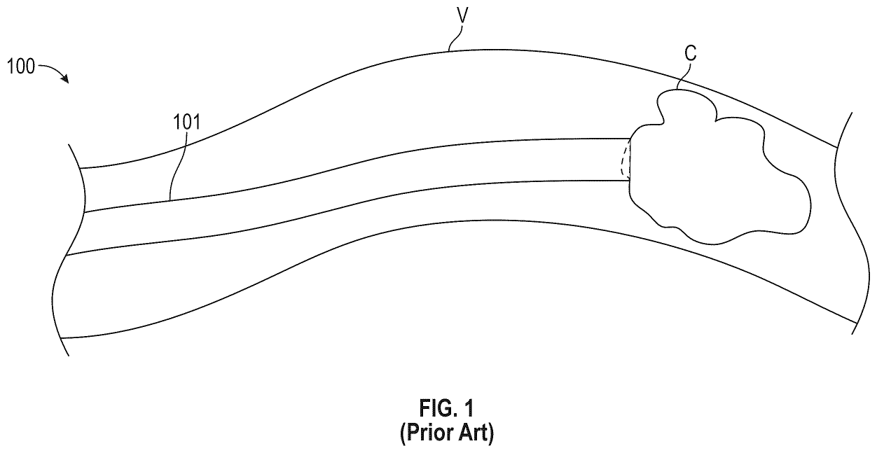

The present technology relates to thrombectomy systems, devices, and methods for treating vascular obstructions, such as vessel occlusions. Some embodiments of the present technology, for example, are directed to a thrombectomy device including a tubular member having a proximal end region configured to be disposed extracorporeally and a distal end region configured to be disposed at an intravascular treatment site. The tubular member can include a sidewall defining a lumen extending from the proximal end region to the distal end region, and a plurality of side openings in the sidewall in the distal end region. The thrombectomy device can further include at least one adjustable flap configured to at least partially cover a side opening of the plurality of side openings of the tubular member. The adjustable flap may have a first end coupled to the tubular member and a free second end. Specific details of several embodiments of the technology are described below with reference to FIGS. 1-8C.

In some embodiments, an aspiration catheter includes a proximal end region and a distal end region configured to be positioned at an intravascular treatment site. The aspiration catheter may further include a sidewall defining a lumen extending from the proximal end region to the distal end region. The aspiration catheter may further include an aspiration opening in the sidewall in the distal end region. The aspiration catheter may further include a cover releasably disposed over the aspiration opening, the cover having a first end coupled to the sidewall and a free second end, the cover adjustable between a closed configuration and an open configuration.

In some embodiments, a method includes disposing a medical device within a vessel at or adjacent a treatment site. The medical device can include a tubular member having a plurality of side openings in a distal region of the tubular member. The medical device can further include at least one adjustable flap coupled to the tubular member, where the adjustable flap is proximate to one of the plurality of side openings, and where the flap has a first configuration in which the flap at least partially covers the side opening and a second configuration in which the flap is biased away from the side opening. The method may further include transitioning the adjustable flap from the first configuration to the second configuration.

The systems and methods of the present technology can provide many advantages compared to conventional devices and techniques for treating vascular obstructions. For instance, the side openings of the tubular member provide for clot engagement at multiple portions of the tubular member. In contrast, conventional aspiration catheters typically rely only on a distal opening through which clotting material is pulled. However, these catheters can be prone to “corking,” in which clotting material completely or substantially blocks the distal opening of the catheter, such that the clot fails to be drawn into a lumen of the catheter. For instance, FIG. 1 illustrates an example aspiration catheter 101 that can be improved using the systems and methods described herein. As shown in FIG. 1, the aspiration catheter 101 has a distal opening 102 that fails to aspirate clot C within a vessel V at a treatment site 100. Accordingly, the aspiration catheter 101 has insufficient overlap with the clot C (e.g., misalignment and/or improper sizing between the distal opening 102 and the clot C) and/or insufficient pressure to remove the clot C, and may instead break apart the clot C, potentially leading to further obstruction and necessitating additional passes (e.g., re-insertion and re-aspiration).

Furthermore, the adjustable flaps described herein can allow for selective opening and/or closing of the side openings of the tubular member to reduce pressure loss and/or unintended material engagement via unused side openings (e.g., side openings that are not actively engaging clotting material). As an example, adjustable flaps that are aligned with (e.g., proximate to) a clot may be opened, whereas adjustable flaps that are not aligned with the clot may remain closed. As will be described further herein, the adjustable flaps may leverage natural intravascular forces, such as blood flow and friction, and therefore provide a passive system for selectively opening and/or closing the side openings.

### I. Overview of Example Treatment Systems and Devices

The present technology provides systems, devices, and methods for removing clotting material from a blood vessel lumen. Although many of the embodiments are described below with respect to devices, systems, and methods for treating a cerebral or intracranial embolism, other applications and other embodiments in addition to those described herein are within the scope of the technology. For example, the thrombectomy systems and methods of the present technology may be used to remove emboli from body lumens other than blood vessels (e.g., the digestive tract, etc.) and/or may be used to remove emboli from blood vessels outside of the brain (e.g., pulmonary, abdominal, cervical, or thoracic blood vessels, or peripheral blood vessels including those within the legs or arms, etc.). In addition, the thrombectomy systems and methods of the present technology may be used to remove luminal obstructions other than clotting material (e.g., plaque, resected tissue, foreign material, etc.).

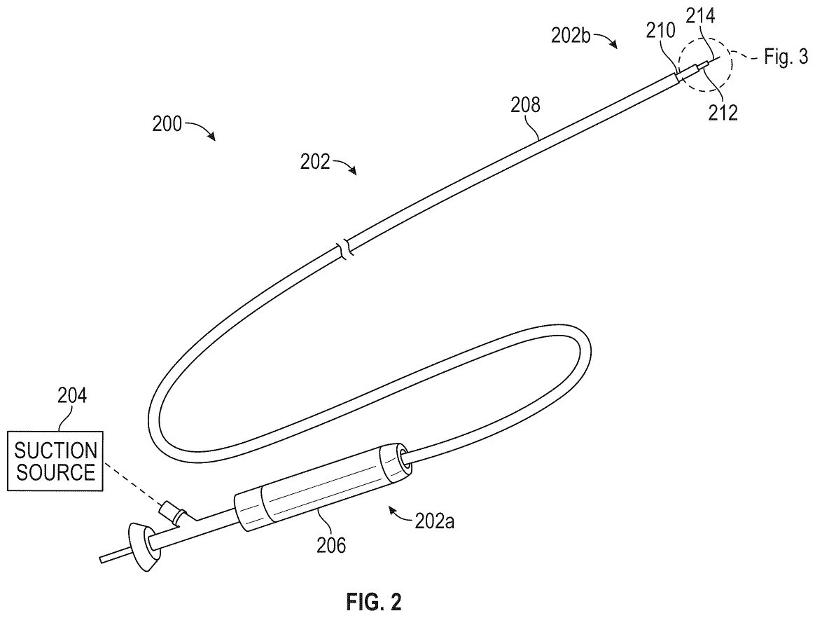

FIG. 2 illustrates a perspective view of a thrombectomy system 200, in accordance with embodiments of the present technology. As shown in FIG. 2., the thrombectomy system 200 can include a medical device assembly 202 and a suction source 204. The medical device assembly 202 includes a proximal portion 202a configured to be coupled to the suction source 204 and a distal portion 202b configured to be intravascularly positioned within a blood vessel (such as an intracranial blood vessel) at a treatment site at or proximate a thrombus. The medical device assembly 202 includes a handle 206 at the proximal portion 202a. A plurality of elongated shafts or tubular members extend between the proximal portion 202a and the distal portion 202b. For example, in some embodiments, such as that shown in FIG. 2, the medical device assembly 202 includes a first or guide catheter 208 (e.g., a large-bore catheter), a distal access catheter 210 configured to be slidably disposed within a lumen of the guide catheter 208, a thrombectomy device 212 in the form of a tubular member (e.g., an aspiration catheter) configured to be slidably disposed within a lumen of the distal access catheter 210, and a guidewire 214 configured to be slidably disposed within a lumen of the thrombectomy device 212. In some embodiments, the medical device assembly 202 does not include one or more of the guide catheter 208, distal access catheter 210, thrombectomy device 212, or the guidewire 214. For instance, where the guide catheter 208 includes a large proximal shaft, there may not be a need for the distal access catheter 210 and/or any intermediate catheters.

In operation, one or more of the guide catheter 208, distal access catheter 210, and thrombectomy device 212 can be used as an aspiration catheter to remove a clot or other material such as plaques or foreign bodies from vasculature of a patient. For example, a vacuum may be applied to a proximal end of the thrombectomy device 212 (e.g., via suction source 204) to draw a clot or other blockage into an inner lumen of the thrombectomy device 212. In some embodiments, the vacuum causes the clot or other blockage to remain attached to the thrombectomy device 212 (e.g., on an outer surface of the thrombectomy device 212). The clot may then be secured by another catheter slidably received over the thrombectomy device 212, e.g., as discussed elsewhere herein. Such aspiration may be used in various medical procedures, such as a medical procedure to treat an ischemic insult, which may occur due to occlusion of a blood vessel (arterial or venous) that deprives brain tissue, heart tissue or other tissues of oxygen-carrying blood.

With continued reference to FIG. 2, in some examples, the thrombectomy device 212 can be configured to access relatively distal locations in a patient including, for example, the middle cerebral artery (MCA), internal carotid artery (ICA), the Circle of Willis, and tissue sites more distal than the MCA, ICA, and the Circle of Willis. The MCA, as well as other vasculature in the brain or other relatively distal tissue sites (e.g., relative to the vascular access point), may be relatively difficult to reach with a tubular member, due at least in part to the tortuous pathway (e.g., comprising relatively sharp twists or turns) through the vasculature to reach these tissue sites. As such, the tubular member may be structurally configured to be relatively flexible, pushable, and relatively kink- and buckle-resistant, so that it may resist buckling when a pushing force is applied to a relatively proximal section of the tubular member to advance the tubular member distally through vasculature, and so that it may resist kinking when traversing around a tight turn in the vasculature. In some examples, the tubular member is configured to substantially conform to the curvature of the vasculature. In addition, in some examples, the tubular member has a column strength and flexibility that allow at least a distal portion of the tubular member to be navigated from a femoral artery, through the aorta of the patient, and into the intracranial vascular system of the patient, e.g., to reach a relatively distal treatment site.

Although primarily described as being used to reach relatively distal vasculature sites, the thrombectomy device 212 may also be configured to be used with other target tissue sites. For example, thrombectomy device 212 may be used to access tissue sites throughout the coronary and peripheral vasculature, the gastrointestinal tract, the urethra, ureters, fallopian tubes, veins and other body lumens.

According to some embodiments, the guide catheter 208 and the distal access catheter 210 can each be formed as additional tubular members extending along and about a central axis and terminating in respective distal ends. According to some embodiments, the distal access catheter 210 is generally constructed to track over the guidewire 214 in the cervical anatomy and into the cerebral vessels associated with the brain and may also be chosen according to several standard designs that are generally available. Accordingly, the distal access catheter 210 can have a length that is at least 125 cm long, and more particularly may be between about 125 cm and about 175 cm long.

The thrombectomy device 212 can be sized and configured to be slidably advanced over the guidewire 214. As noted above, the thrombectomy device 212 can be coupled at a proximal portion to a suction source 204 such as a pump or syringe in order to supply negative pressure to a treatment site. In various embodiments, the thrombectomy device 212 can have a length that is at least 125 cm long, and more particularly may be between about 125 cm and about 175 cm long. In some embodiments, the thrombectomy device 212 can be an aspiration catheter. The thrombectomy device 212 can have a lumen diameter of a between about 0.05″ and about 0.09″, for example about 0.045″, about 0.055″, about 0.061″, about 0.068″, or about 0.071″ lumen diameter. The distal access catheter 210 can have a maximum outer diameter of between about 0.06″ to about 0.1″, for example about 0.083″, or about 0.0855″. In some embodiments, a distal tip of the distal access catheter 210 can have an outer diameter of between 0.031″ to 0.037″. Other designs and dimensions are contemplated.

The guide catheter 208 can be sized and configured to slidably receive both the distal access catheter 210 and the thrombectomy device 212 therethrough. In some embodiments, the guide catheter 208 is a balloon-guide catheter having an inflatable balloon or other expandable member that can be used to anchor the guide catheter 208 with respect to a surrounding vessel. In operation the guide catheter 208 can first be advanced through a vessel and then a balloon can be expanded to anchor the guide catheter 208 in place and/or arrest blood flow from areas proximal of the balloon. Next, the distal access catheter 210 and the thrombectomy device 212 can be advanced together through the guide catheter 208 until they each extend distally beyond the distal end of the guide catheter 208. Suction can then be applied to aspirate the treatment site.

According to some embodiments, the bodies of the catheter 208, distal access catheter 210, and/or thrombectomy device 212 can be made from silicone and/or various thermoplastics, e.g., polytetrafluoroethylene (PTFE or TEFLON®), fluorinated ethylene propylene (FEP), high-density polyethylene (HDPE), polyether ether ketone (PEEK), etc., which can optionally be lined on the inner surface of the catheters and/or tubular member or an adjacent surface with a hydrophilic material such as polyvinylpyrrolidone (PVP) or some other plastic coating. Additionally, either surface can be coated with various combinations of different materials, depending upon the desired results. As described in more detail below, some or all of the thrombectomy device 212 can be formed of a metallic material, such as Nitinol, stainless steel, or other suitable material. In some examples, the thrombectomy device 212 can include a laser-cut hypotube having a pattern of cut voids (e.g. spiral cut, separated slot cuts, or other suitable pattern) formed in its sidewall along at least a portion of its length. In at least some embodiments, the thrombectomy device 212 can have a laser cut pattern to achieve the desired mechanical characteristics (e.g., column strength, flexibility, kink-resistance, etc.).

In various embodiments, the guidewire 214 can be a solid pushwire or guidewire. Additionally or alternatively, the guidewire 214 can instead include a hollow wire, hypotube, braid, coil, or other suitable member(s), or a combination of wire(s), tube(s), braid(s), coil(s), etc. In some embodiments, the guidewire 214 can be made of stainless steel (e.g., 304 SS), Nitinol, and/or other alloy.

In some embodiments, the thrombectomy system 200 is configured to be deployed at an intravascular treatment site (e.g., at or adjacent to a thrombus). A guidewire 214 slidably extends through a lumen of the thrombectomy device 212, which in turn slidably extends through of a lumen of the surrounding catheter 210. The guidewire 214 can be configured to assist in delivery of the thrombectomy device 212 to the intravascular treatment site. The guidewire 214 can then be removed after the thrombectomy device 212 is positioned at the intravascular treatment site. As noted elsewhere herein, the thrombectomy system 200 can include a suction source 204 such that when suction is applied, the thrombectomy device 212 is configured to engage clotting material.

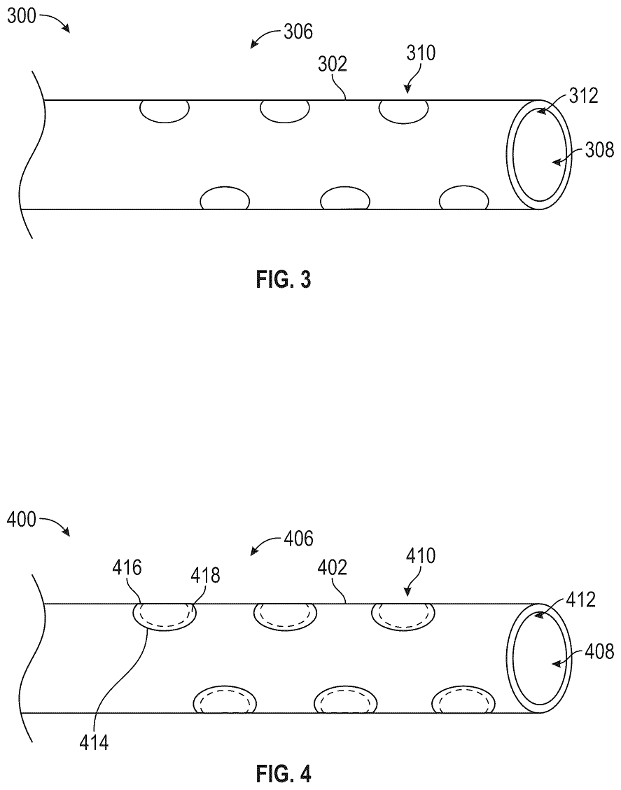

FIG. 3 illustrates a side view of an example distal portion 202b of the thrombectomy system 200 of FIG. 2, in accordance with embodiments of the present technology. Specifically, FIG. 3 illustrates a distal end region 300 of a thrombectomy device in the form of a tubular member 302. The tubular member 302 can include a proximal portion (not shown in FIG. 3) and a distal portion 306. The tubular member 302 can define a lumen 308 extending along the length of the tubular member 302 between the proximal portion and the distal portion 306. In some implementations, the proximal portion of the tubular member 302 is configured to be coupled to a suction source (not depicted) to aspirate clotting material via the lumen 308 of the tubular member 302. For instance, the tubular member 302 can include one or more side openings 310. The side openings 310 can be configured to fluidically couple the lumen 308 with an environment surrounding the tubular member 302 (e.g., a blood vessel).

The side openings 310 can include any variety of geometries. For instance, the side openings 310 can be or include circular, straight, arcuate, curved, semi-circular, or semi-elliptical shapes. The side openings 310 may optionally be or include complex shapes, such as zig-zag, undulating, undulating, serpentine, sinusoidal, or a combination thereof. In some implementations, the side openings 310 can take the form of windows, apertures, voids, cuts, or other such structures that allow fluid to pass therethrough. The side openings 310 can be arranged with longitudinal spacing and/or radial spacing. For instance, as shown in FIG. 3, the side openings 310 can define a first set of 3 longitudinally arranged openings on a first radial side of the tubular member 302 and a second set of 3 longitudinally arranged openings on an opposing radial side of the tubular member 302. The first set and second set of openings may be offset from one another longitudinally. Further, side openings 310 may be arranged in other configurations, such as in a spiral around the tubular member 302. Other arrangements are contemplated.

The tubular member 302 can further include a distal opening 312 in the distal portion 306. In some embodiments, the distal opening 312 is configured to permit passage of the guidewire 214 therethrough, allowing the tubular member 302 to be slidably advanced over the guidewire 214. A distal end of the side openings 310 and the distal opening 312 can be separated from one another along the longitudinal axis of the tubular member 302 by at least 1 mm, 5 mm, 10 mm, 20 mm, 50 mm, 100 mm, 1 cm, 5 cm, 10 cm, etc.

### A. Select Examples of Thrombectomy Devices Having Adjustable Flaps

In some embodiments, the side openings of the thrombectomy device can be selectively opened and closed. For instance, a side opening can have an open configuration in which the side opening is in fluid communication with the surrounding environment, such as to engage clot material within the surrounding environment. However, it may be desirable to close the side opening (e.g., a closed configuration) when not engaging clot material, such as to prevent leakage of air or liquid through the side opening.

In some embodiments, the side opening can be opened and/or closed via at least one adjustable flap. The adjustable flap may be positioned proximate to the side opening, such as on an external surface of the thrombectomy device. The adjustable flap may have a first end coupled to a tubular member of the thrombectomy device and a free second end. Further, the adjustable flap may have a first configuration in which the free second end is biased toward the sidewall of the tubular member to at least partially cover the side opening and a second configuration in which the free second end is biased away from the sidewall of the tubular member.

FIG. 4 illustrates a side view of an example distal end region 400 of the thrombectomy device 212 of the thrombectomy system 200 of FIG. 2, in accordance with embodiments of the present technology. The distal end region 400 can be generally similar to any of the distal end regions discussed herein, such as the distal end region 300 of FIG. 3. For instance, the distal end region 400 can include a tubular member 402 having a proximal portion (not shown in FIG. 4), a distal portion 406, and a lumen 408 defined therebetween. Further, the tubular member 402 can include a plurality of side openings 410 and a distal opening 412. In some embodiments, the tubular member 402 is configured to be coupled to a suction source (e.g., the suction source 204 of the thrombectomy system 200 of FIG. 2), and the tubular member 402 can aspirate clotting material and/or vascular debris via the side openings 410 and/or the distal opening 412.

In some embodiments, the distal end region 400 includes at least one adjustable flap 414. The adjustable flap 414 can be configured to at least partially cover a side opening of the plurality of side openings 410. For instance, the adjustable flap 414 may include a first end 416 coupled to the tubular member 402 and a free second end 418. The first end 416 can be coupled to the tubular member 402 via one or more of adhesives, bonding, welding, etc. Alternatively or in addition, the adjustable flap 414 may be integrally formed with the tubular member 402. For instance, the adjustable flap 414 may be laser cut into the tubular member 402. The free second end 418 may rest flush against the tubular member 402. Optionally, the free second end 418 may be releasably coupled to the tubular member 402. For instance, the free second end 418 may have a loose adhesive configured to releasably couple the free second end 418 to the tubular member 402. The loose adhesive can be released, for instance, in response to force and/or pressure applied to the tubular member 402.

The adjustable flap 414 can have a first configuration in which the free second end 418 is biased toward the sidewall of the tubular member 402 and a second configuration in which the free second end 418 is biased away from the sidewall of the tubular member 402. In some embodiments, the adjustable flap 414 transitions from the first configuration to the second configuration in response to blood contacting the free second end 418 (e.g., blood flow imparts force on the free second end 418). Alternatively or in combination, the adjustable flap 414 can transition from the first configuration to the second configuration in response to positive pressure applied to the lumen 408 of the tubular member 402. Optionally, the adjustable flap 414 can transition from the first configuration to the second configuration in response to frictional engagement between the free second end 418 and clot material at the treatment site, as will be described further herein.

In some embodiments, the adjustable flap 414 is configured to transition from the second configuration to the first configuration in response to negative pressure. For instance, aspiration applied to the lumen 408 of the tubular member 402 may cause the free second end 418 of the adjustable flap 414 to be urged toward the tubular member 402. This may be useful, for instance, in sealing the side opening 410 when the side opening 410 is not actively engaging clotting material.

The adjustable flaps 414 can be substantially rectangular, triangular, round, circular, oblong, or any other suitable geometry. In some embodiments, the adjustable flaps 414 are sized to fully cover corresponding side openings 410 in the tubular member 402. For instance, as shown in FIG. 4, the adjustable flaps 414 have a greater area than the side openings 410. However, the adjustable flaps 414 may alternatively be sized to only partially cover the corresponding side openings 410. Optionally, some of the adjustable flaps 414 may be configured to cover a subset of the side openings 410. For instance, an adjustable flap 414 may cover two or more side openings 410. The adjustable flaps 414 can include any suitable number of flaps such as 1, 2, 3, 4, 5, 6, 7, 8, 9, or 10 flaps.

In some embodiments, the adjustable flap 414 is flexible. For instance, the adjustable flaps 414 may include a sufficiently high elasticity and/or sufficiently low rigidity to fold, bend, deflect, furl, etc. In some embodiments, the adjustable flaps 414 comprise soft and/or stretchable materials such as thermoplastics and silicone, and/or metals such as nickel titanium. Further, the adjustable flaps 414 can be radiopaque. This may be useful, for example, in positioning and/or manipulating the tubular member 402 at the treatment site.

The adjustable flaps 414 can be spaced apart circumferentially. For instance, in some embodiments, neighboring adjustable flaps 414 are spaced apart circumferentially by an angle ranging between 0 degrees and 30 degrees, 30 degrees and 60 degrees, 60 degrees and 90 degrees, 90 degrees and 120 degrees, 120 degrees and 150 degrees, 150 degrees and 180 degrees, 180 degrees and 210 degrees, 210 degrees and 240 degrees, 240 degrees and 270 degrees, 270 degrees and 300 degrees, 300 degrees and 330 degrees, or 330 degrees and 360 degrees.

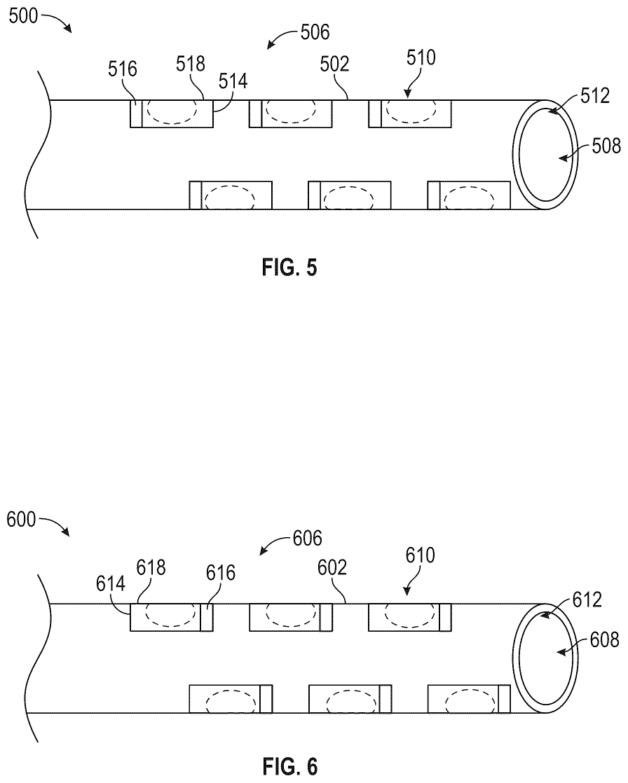

FIG. 5 illustrates a side view of an example distal end region 500 of the thrombectomy device 212 of the thrombectomy system 200 of FIG. 2, in accordance with embodiments of the present technology. The distal end region 500 can be generally similar to any of the distal end regions discussed herein, such as the distal end region 400 of FIG. 4. For instance, the distal end region 500 can include a tubular member 502 having a proximal portion (not shown in FIG. 5), a distal portion 506, and a lumen 508 defined therebetween. Further, the tubular member 502 can include a plurality of side openings 510 and a distal opening 512. In some embodiments, the tubular member 502 is configured to be coupled to a suction source (e.g., the suction source 204 of the thrombectomy system 200 of FIG. 2), and the tubular member 502 can aspirate clotting material and/or vascular debris via the side openings 510 and/or the distal opening 512.

In some embodiments, the distal end region 500 includes at least one adjustable flap 514. The adjustable flap 514 can be configured to at least partially cover a side opening 510 of the plurality of side openings 510. For instance, the adjustable flap 514 may include a first end 516 coupled to the tubular member 502 and a free second end 518. In some embodiments, the first end 516 is proximal to the free second end 518. For instance, the first end 516 may be coupled to a proximal segment of the tubular member 502 (e.g., a portion of the tubular member 502 proximal to the side opening 510), and the free second end 518 may extend distally.

In the illustrated embodiment, the adjustable flap 514 has a rectangular geometry and is configured to fully cover the side opening 510. Some or all of the first end 516 is directly coupled to the tubular member 502. In some embodiments, the first end 516 can be coupled to the tubular member 502 using one or more of adhesives, bonding, welding, etc. Additionally or alternatively, the first end 516 may be integrally formed with the tubular member 502. For instance, the adjustable flap 514 may be laser cut into the tubular member 502 with the first end 516 maintaining continuity with the rest of the tubular member 502.

In some implementations, the free second end 518 may be flush with the tubular member 502. For instance, the free second end 518 may contact the tubular member 502 without being directly coupled to the tubular member 502. Alternatively, the free second end 518 may be releasably coupled to the tubular member 502. For instance, the free second end 518 may be releasably coupled to the tubular member 502 via an adhesive.

The adjustable flap 514 can have a first configuration in which the free second end 518 is biased toward the sidewall of the tubular member 502 and a second configuration in which the free second end 518 is biased away from the sidewall of the tubular member 502. The adjustable flap 514 can transition from the first configuration to the second configuration in response to frictional engagement of the free second end 518 with clotting material. For instance, the tubular member 502 can be advanced within a body lumen until the clotting material is adjacent to and/or near the free second end 518. The tubular member 502 can then be manipulated (e.g., rotated, advanced, retracted) until the free second end 518 pushes against the clotting material (or vice versa), and the free second end 518 is deflected away from the sidewall of the tubular member 502.

Alternatively or in addition, the adjustable flap 514 can transition from the first configuration to the second configuration in response to positive pressure applied to the tubular member 502. For instance, a suction source (e.g., the suction source 204 of FIG. 2) may provide positive pressure to the lumen 508 of the tubular member 502. The positive pressure can cause the free second end 518 to deflect away from the sidewall of the tubular member 502.

Alternatively or in addition, the adjustable flap 514 can transition from the first configuration to the second configuration in response to blood flow within the body lumen. For instance, as the tubular member 502 is distally advanced, the free second end 518 of the adjustable flap 514 may face resistance from blood, and the blood may cause the free second end 518 to deflect away from the sidewall of the tubular member 502.

In some embodiments, the adjustable flap 514 is configured to transition from the second configuration to the first configuration in response to negative pressure. For instance, aspiration applied to the lumen 508 of the tubular member 502 may cause the free second end 518 of the adjustable flap 514 to be urged toward the tubular member 502. This may be useful, for instance, in sealing the side opening 510 when the side opening 510 is not actively engaging clotting material.

FIG. 6 illustrates a side view of an example distal end region 600 of the thrombectomy device 212 of the thrombectomy system 200 of FIG. 2, in accordance with embodiments of the present technology. The distal end region 600 can be generally similar to any of the distal end regions discussed herein, such as the distal end region 400 of FIG. 4. For instance, the distal end region 600 can include a tubular member 602 having a proximal portion (not shown in FIG. 6), a distal portion 606, and a lumen 608 defined therebetween. Further, the tubular member 602 can include a plurality of side openings 610 and a distal opening 612. In some embodiments, the tubular member 602 is configured to be coupled to a suction source (e.g., the suction source 204 of the thrombectomy system 200 of FIG. 2), and the tubular member 602 can aspirate clotting material and/or vascular debris via the side openings 610 and/or the distal opening 612.

In some embodiments, the distal end region 600 includes at least one adjustable flap 614. The adjustable flap 614 can be configured to at least partially cover a side opening of the plurality of side openings 610. For instance, the adjustable flap 614 may include a first end 616 coupled to the tubular member 602 and a free second end 618. In some embodiments, the first end 616 is distal to the free second end 618. For instance, the first end 616 may be coupled to a distal segment of the tubular member 602 (e.g., a portion of the tubular member 602 distal to the side opening 610), and the free second end 618 may extend toward the proximal portion of the tubular member 602.

In the illustrated embodiment, the adjustable flap 614 has a rectangular geometry and is configured to fully cover the side opening 610. Some or all of the first end 616 is directly coupled to the tubular member 602. In some embodiments, the first end 616 can be coupled to the tubular member 602 using one or more of adhesives, bonding, welding, etc. Additionally or alternatively, the first end 616 may be integrally formed with the tubular member 602. For instance, the adjustable flap 614 may be laser cut into the tubular member 602 with the first end 616 maintaining continuity with the rest of the tubular member 602.

In some implementations, the free second end 618 may be flush with the tubular member 602. For instance, the free second end 618 may contact the tubular member 602 without being directly coupled to the tubular member 602. Alternatively, the free second end 618 may be releasably coupled to the tubular member 602. For instance, the free second end 618 may be releasably coupled to the tubular member 602 via an adhesive.

The adjustable flap 614 can have a first configuration in which the free second end 618 is biased toward the sidewall of the tubular member 602 and a second configuration in which the free second end 618 is biased away from the sidewall of the tubular member 602. The adjustable flap 614 can transition from the first configuration to the second configuration in response to frictional engagement of the free second end 618 with clotting material. For instance, the tubular member 602 can be advanced in a body lumen past clotting material and then retracted until the clotting material is adjacent to and/or near the free second end 618. The tubular member 602 can then be manipulated (e.g., rotated, advanced, retracted) until the free second end 618 pushes against the clotting material (or vice versa), and the free second end 618 is deflected away from the sidewall of the tubular member 602.

Alternatively or in addition, the adjustable flap 614 can transition from the first configuration to the second configuration in response to positive pressure applied to the tubular member 602. For instance, a suction source (e.g., the suction source 204 of FIG. 2) may provide positive pressure to the lumen 608 of the tubular member 602. The positive pressure can cause the free second end 618 to deflect away from the sidewall of the tubular member 602.

Alternatively or in addition, the adjustable flap 614 can transition from the first configuration to the second configuration in response to blood flow within the body lumen. For instance, as the tubular member 602 is distally advanced, the free second end 618 of the adjustable flap 614 may face resistance from blood, and the blood may cause the free second end 618 to deflect away from the sidewall of the tubular member 602.

In some embodiments, the adjustable flap 614 is configured to transition from the second configuration to the first configuration in response to negative pressure. For instance, aspiration applied to the lumen 608 of the tubular member 602 may cause the free second end 618 of the adjustable flap 614 to be urged toward the tubular member 602. This may be useful, for instance, in sealing the side opening 610 when the side opening 610 is not actively engaging clotting material.

### II. Example Treatment Methods

The thrombectomy devices and systems provided herein can be used in treating a variety of vessel occlusions. Although the methods are described herein primarily with reference to a thrombectomy device with Figure-specific reference numbers for clarity, it should be understood that the methods described herein may additionally or alternatively be performed with any suitable variation of thrombectomy devices in accordance with the present technology (e.g., a thrombectomy device having distal end regions 300, 400, etc.), such as those described above with respect to FIGS. 2-6.

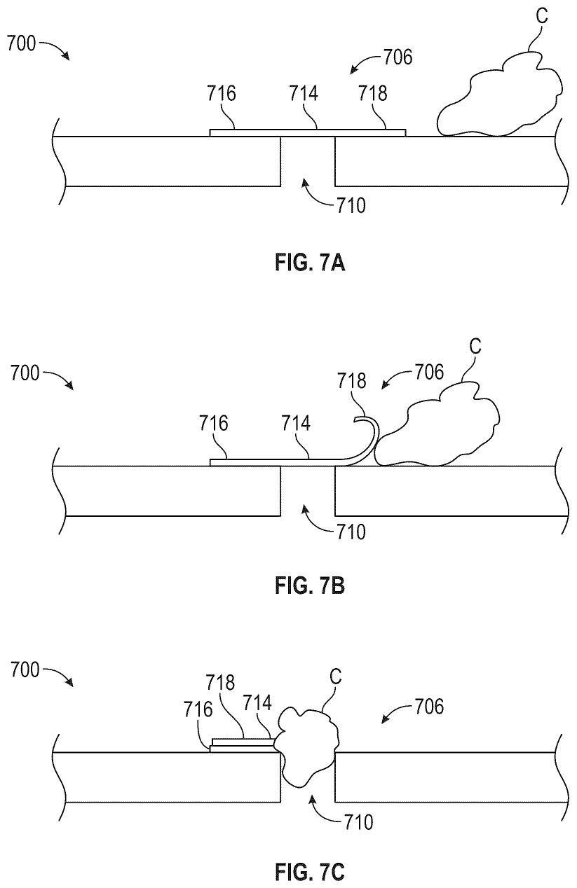

FIGS. 7A-7C depict the deployment of a thrombectomy device 700 adjacent to or near a clot C within a blood vessel. Thrombectomy device 700 can include a tubular member 702 (only one side of a distal end region of the tubular member 702 is shown for clarity; it should be understood that the tubular member 702 may include another side opposing the side shown with similar structure and/or function). The thrombectomy device 700 can further include an adjustable flap 714 coupled to the tubular member 702. In the illustrated embodiment, a first end 716 of the adjustable flap 714 is coupled to a proximal segment of the tubular member 702 (e.g., a portion of the tubular member 702 proximal to the side opening 710) and a free second end 718 extends over a length of a distal segment of the tubular member 702 (e.g., a portion of the tubular member 702 distal to the side opening 710). The adjustable flap 714 can be configured to at least partially cover the side opening 710 of the tubular member 702.

Referring now to FIG. 7A, the thrombectomy device 700 can be distally advanced until the clot C is adjacent to or near the distal segment 706 of the tubular member 702. In some embodiments, the thrombectomy device 700 is advanced over a guidewire (not depicted), or is navigated to the treatment site using various other medical devices (e.g., catheters), as discussed above with respect to the thrombectomy system 200 of FIG. 2. During delivery, the adjustable flap 714 may be in a first configuration in which the free second end 718 is biased toward to the sidewall of the tubular member 702 such that the majority or all of the side opening 710 is covered by the adjustable flap 714. This may be useful in preventing vascular debris and/or unintended materials from entering the tubular member 702 prior to reaching the treatment site.

Referring now to FIG. 7B, after the thrombectomy device 700 is distally advanced to the treatment site, the thrombectomy device 700 can be further advanced such that the free second end 718 of the adjustable flap 714 abuts the clot C. In some embodiments, this may involve rotating, retracting, and/or advancing the thrombectomy device 700 until the adjustable flap 714 abuts the clot C. Upon further advancement of the thrombectomy device 700 within the vessel, frictional engagement between the clot C and the adjustable flap 714 may cause the adjustable flap 714 to transition to a second configuration in which the free second end 718 is biased away (e.g., deflected, perturbed) from the sidewall of the tubular member 702. For instance, a proximal portion of the clot C may displace the free second end 718 of the adjustable flap 714. In some embodiments, the free second end 718 includes a lip, ridge, bump, protrusion, or other such structure that facilitates engagement with the clot C. Alternatively or in addition, the free second end 718 may include one or more material properties that facilitate engagement with the clot C. For instance, the free second end 718 may have a greater elasticity than the first end 716.

Turning now to FIG. 7C, the transitioning of the free second end 718 of the adjustable flap 714 from the first configuration to the second configuration may include folding the free second end 718 over the first end 716 in the proximal segment of the tubular member 702. This can cause a majority or all of the side opening 710 to be exposed to the surrounding environment. Optionally, the free second end 718 may be secured to the first end 716, such as via adhesives. However, the free second end 718 may also remain free to move following displacement.

With the side opening 710 at least partially uncovered, aspiration forces may be applied to the tubular member 702. For instance, negative pressure can be applied to the tubular member 702 using a suction source attached to a proximal portion of the tubular member 702. The negative pressure can cause the clot C to be engaged with and/or aspirated into the tubular member 702 via the side opening 710. After engagement and/or aspiration of the clot C, the tubular member 702 can be retracted from the vessel to remove the clot C from the treatment site. For instance, the tubular member 702 may be retracted while maintaining negative pressure.

In some embodiments, the thrombectomy systems and devices described herein may include a plurality of side openings. However, it may be advantageous to engage clotting material through only a subset of the side openings while keeping unengaged side openings covered. This may be beneficial, for instance, to reduce pressure loss and/or accidental material engagement through the unengaged side openings. In operation, adjustable covers that open only upon engagement with clot material C can beneficially remain closed unless and until they engage clot material. For a device having a plurality of side openings each with respective covers, only those covers which abut and engage with the clot C will evert into an open configuration and allow engagement between the now-exposed underlying side opening and the clot C, while other side openings remain covered. This can reduce leakage through non-engaged side openings and increase the suction force that is applied to the clot C.

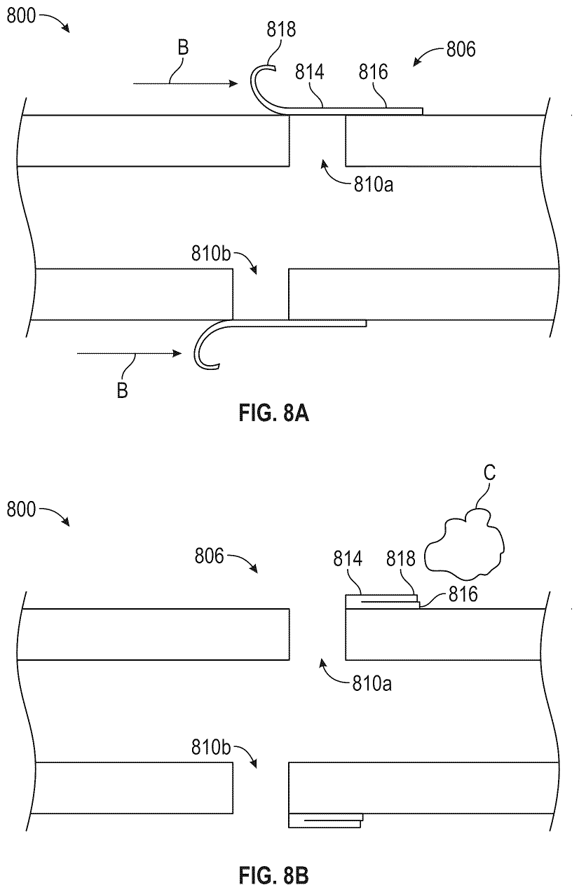

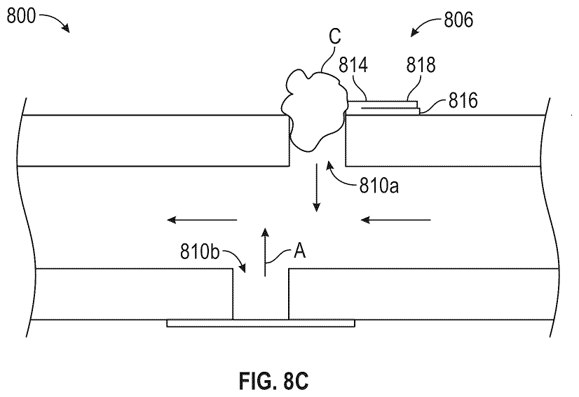

FIGS. 8A-8C depict the deployment of a thrombectomy device 800 adjacent to or near a clot C. Thrombectomy device 800 can include a tubular member 802 (both sides of the tubular member 802 are shown in this illustration). The thrombectomy device 800 can further include a plurality of adjustable flaps 814 coupled to the tubular member 802. In the illustrated embodiment, a first end 816 of each adjustable flap 814 is coupled to a distal segment of the tubular member 802, and a free second end 818 of each adjustable flap 814 extends over a length of a proximal segment of the tubular member 802. Each adjustable flap 814 can be configured to at least partially cover a respective side opening of the tubular member 802. For clarity, the side openings are shown to include an upper side opening 810a and a lower side opening 810b. However, it should be understood that the tubular member 802 may include additional side openings and the positioning of the side openings may vary across thrombectomy devices.

Referring now to FIG. 8A, the thrombectomy device 800 can be distally advanced to a treatment site within a blood vessel. The thrombectomy device 800 can be distally advanced with the adjustable flaps 814 in a first configuration in which the free second ends 818 contact the proximal segment of the tubular member 802 (e.g., a portion of the tubular member 802 proximal to the side opening 810a). Optionally, the thrombectomy device 800 may be delivered to the treatment site with the free second ends 818 at least partially furled (e.g., deflected) away from the tubular member 802. However, the free second ends 818 may additionally or alternatively be configured to be flush against the proximal segment of the tubular member 802 during device positioning.

In some embodiments, blood flow B may be configured to abut the free second ends 818. The blood flow B may be configured to cause the adjustable flaps 814 to transition from the first configuration to a second configuration in which the free second ends 818 no longer contact the proximal segment of the tubular member 802. For instance, the blood flow B may cause the free second ends 818 to furl (e.g., deflect) away from the proximal segment, as partially shown.

Turning now to FIG. 8B, the blood flow B may cause the free second ends 818 of the adjustable flaps 814 to fold over the first ends 816 of the adjustable flaps 814 in the distal segment 806 of the tubular member 802 (e.g., the portion of the tubular member 802 distal to the side opening 810a). Optionally, the free second ends 818 may be secured to the first ends 816, such as with the use of adhesives on the first ends 816 and/or free second ends 818. Additionally or alternatively, the free second ends 818 may be secured to the first ends 816 via forces imparted by the blood flow B.

The thrombectomy device 800 can be distally advanced until the clot C is adjacent to and/or near the distal segment 806 of the tubular member 802. In some embodiments, the clot C is adjacent to and/or near only one side of the tubular member 802 (shown here as an upper side of the tubular member 802). The thrombectomy device 800 can be distally advanced until the clot C is close to upper side opening 810a.

Referring now to FIG. 8C, with the upper side opening 810a at least partially uncovered, aspiration forces A can be applied to the tubular member 802. For instance, negative pressure can be applied to the tubular member 802 using a suction source attached to a proximal portion of the tubular member 802. The negative pressure can cause the clot C to be engaged with and/or aspirated into the tubular member 802 via the upper side opening 810a. At the same time, the negative pressure can cause adjustable flaps 814 that are not adjacent to and/or near the clot C to close. Stated differently, unused side openings (e.g., lower side opening 810b) can be covered by respective adjustable flaps 814, since the unused side openings are not engaged by clotting material. As a result, the lower side opening 810b is sealed and aspiration pressure is not lost through the lower side opening 810b. Moreover, the sealing (e.g., at least partially covering the lower side opening 810b via a respective adjustable flap 814) may enhance aspiration pressure through the upper side opening 810a.

After engagement and/or aspiration of the clot C through the upper side opening 810a, the tubular member 802 can be retracted from the vessel to remove the clot C from the treatment site. For instance, the tubular member 802 can be retracted while maintaining negative pressure. As shown, the negative pressure may also maintain sealing of the lower side opening 810b.

Optionally, the methods of the present technology can be performed under fluoroscopy such that at least some portions of the thrombectomy device can be visualized by a physician to ensure proper placement of the thrombectomy device. For example, the thrombectomy device can include one or more radiopaque portions. The one or more radiopaque portions can be visualized using fluoroscopy and/or other suitable imaging techniques to assist in positioning the thrombectomy device.

### EXAMPLES

The subject technology is illustrated, for example, according to various aspects described below, including with reference to FIGS. 1-8C. Various examples of aspects of the subject technology are described as numbered examples (1, 2, 3, etc.) for convenience. These are provided as examples and do not limit the subject technology.

### Example 1

A thrombectomy device comprising: a tubular member having a proximal end region configured to be disposed extracorporeally and a distal end region configured to be disposed at an intravascular treatment site, the tubular member comprising: a sidewall defining a lumen extending from the proximal end region to the distal end region, and a plurality of side openings in the sidewall in the distal end region; and at least one adjustable flap configured to at least partially cover a side opening of the plurality of side openings of the tubular member, wherein the adjustable flap comprises a first end coupled to the tubular member and a free second end.

### Example

The thrombectomy device of any one of the preceding Examples, wherein the adjustable flap has a first configuration in which the free second end is biased toward the sidewall of the tubular member to at least partially cover the side opening and a second configuration in which the free second end is biased away from the sidewall of the tubular member.

### Example 3

The thrombectomy device of any one of the preceding Examples, wherein the adjustable flap is configured to transition from the first configuration to the second configuration in response to blood flow contacting the free second end.

### Example 4

The thrombectomy device of any one of the preceding Examples, wherein the adjustable flap is configured to transition from the first configuration to the second configuration in response to positive pressure applied to the tubular member lumen.

### Example 5

The thrombectomy device of any one of the preceding Examples, wherein the adjustable flap is configured to transition from the first configuration to the second configuration in response to frictional engagement between the free second end and clot material at the treatment site.

### Example 6

The thrombectomy device of any one of the preceding Examples, wherein the adjustable flap is configured to transition from the second configuration to the first configuration in response to negative pressure applied to the tubular member.

### Example 7

The thrombectomy device of any one of the preceding Examples, wherein, when the negative pressure is applied to the tubular member, at least one of the side openings remains uncovered.

### Example 8

The thrombectomy device of any one of the preceding Examples, wherein the adjustable flap is in the first configuration during delivery of the thrombectomy device to the treatment site.

### Example 9

The thrombectomy device of any one of the preceding Examples, wherein the adjustable flap is in the second configuration during delivery of the thrombectomy device to the treatment site.

### Example 10

The thrombectomy device of any one of the preceding Examples, wherein the first end is proximal of the free second end.

### Example 11

The thrombectomy device of any one of the preceding Examples, wherein the first end is distal of the free second end.

### Example 12

The thrombectomy device of any one of the preceding Examples, wherein the adjustable flap is integrally formed with the tubular member.

### Example 13

The thrombectomy device of any one of the preceding Examples, wherein the side openings are circumferentially arranged around the tubular member.

### Example 14

The thrombectomy device of any one of the preceding Examples, wherein the adjustable flap is rounded.

### Example 15

The thrombectomy device of any one of the preceding Examples, wherein the thrombectomy device is configured to engage a thrombus at the treatment site in response to negative pressure.

### Example 16

An aspiration catheter comprising: a proximal end region, a distal end region configured to be positioned at an intravascular treatment site; a sidewall defining a lumen extending from the proximal end region to the distal end region; an aspiration opening in the sidewall in the distal end region; and a cover releasably disposed over the aspiration opening, the cover having a first end coupled to the sidewall and a free second end, the cover adjustable between a closed configuration and an open configuration.

### Example 17

A method comprising: disposing a medical device within a vessel at or adjacent a treatment site, the medical device comprising: a tubular member, the tubular member having a plurality of side openings in a distal region of the tubular member, and at least one adjustable flap coupled to the tubular member, wherein the adjustable flap is proximate to a one of the plurality of side openings, and wherein the flap has a first configuration in which the flap at least partially covers the side opening and a second configuration in which the flap is biased away from the side opening; and transitioning the adjustable flap from the first configuration to the second configuration.

### Example 18

The method of any one of the preceding Examples, wherein the adjustable flap is transitioned from the first configuration to the second configuration in response to blood flow contacting a proximal end of the adjustable flap.

### Example 19

The method of any one of the preceding Examples, further comprising applying positive pressure to the tubular member, wherein the positive pressure causes the adjustable flap to transition from the first configuration to the second configuration.

### Example 20

The method of any one of the preceding Examples, further comprising distally advancing the medical device toward a thrombus at the treatment site, wherein the distal advancement causes the thrombus to abut against a distal end of the adjustable flap, thereby transitioning the adjustable flap from the first configuration to the second configuration.

### Example 21

The method of any one of the preceding Examples, further comprising applying negative pressure to the tubular member, thereby causing one or more adjustable flaps to transition from the second configuration to the first configuration.

### Example 22

The method of any one of the preceding Examples, wherein the one or more adjustable flaps includes the adjustable flap.

### Example 23

The method of any one of the preceding Examples, wherein the medical device is disposed at the treatment site with the adjustable flap in the first configuration.

### Example 24

The method of any one of the preceding Examples, wherein the medical device is disposed at the treatment site with the adjustable flap in the second configuration.

### Example 25

The method of any one of the preceding Examples, wherein the adjustable flap comprises a first end coupled to the tubular member and a free second end.

### Example 26

The method of any one of the preceding Examples, wherein the first end is proximal of the free second end.

### Example 27

The method of any one of the preceding Examples, wherein the first end is distal of the free second end.

### Example 28

The method of any one of the preceding Examples, wherein the adjustable flap is integrally formed with the tubular member.

### Example 29

The method of any one of the preceding Examples, wherein the side openings are circumferentially arranged around the tubular member.

### Example 30

The method of any one of the preceding Examples, wherein the adjustable flap is round.

### Example 31

The method of any one of the preceding Examples, further comprising applying negative pressure to the tubular member to engage clotting material at the treatment site via at least some of the side openings.

### CONCLUSION

Although many of the embodiments are described above with respect to systems, devices, and methods for treating vessel occlusions in the brain, the technology is applicable to other applications and/or other approaches, such as vessel occlusions elsewhere in the body. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-8C.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A thrombectomy device comprising:
a tubular member having a proximal end region configured to be disposed extracorporeally and a distal end region configured to be disposed at an intravascular treatment site, the tubular member comprising:
a sidewall defining a lumen extending from the proximal end region to the distal end region, and
a plurality of side openings in the sidewall in the distal end region; and
at least one adjustable flap configured to at least partially cover a side opening of the plurality of side openings of the tubular member, wherein the adjustable flap comprises a first end coupled to the tubular member and a free second end.

2. The thrombectomy device of claim 1, wherein the adjustable flap has a first configuration in which the free second end is biased toward the sidewall of the tubular member to at least partially cover the side opening and a second configuration in which the free second end is biased away from the sidewall of the tubular member.

3. The thrombectomy device of claim 2, wherein the adjustable flap is configured to transition from the first configuration to the second configuration in response to blood flow contacting the free second end.

4. The thrombectomy device of claim 2, wherein the adjustable flap is configured to transition from the first configuration to the second configuration in response to positive pressure applied to the tubular member lumen.

5. The thrombectomy device of claim 2, wherein the adjustable flap is configured to transition from the first configuration to the second configuration in response to frictional engagement between the free second end and clot material at the treatment site.

6. The thrombectomy device of claim 2, wherein the adjustable flap is configured to transition from the second configuration to the first configuration in response to negative pressure applied to the tubular member.

7. The thrombectomy device of claim 6, wherein, when the negative pressure is applied to the tubular member, at least one of the side openings remains uncovered.

8. The thrombectomy device of claim 2, wherein the adjustable flap is in the first configuration during delivery of the thrombectomy device to the treatment site.

9. The thrombectomy device of claim 2, wherein the adjustable flap is in the second configuration during delivery of the thrombectomy device to the treatment site.

10. The thrombectomy device of claim 1, wherein the first end is proximal of the free second end.

11. The thrombectomy device of claim 1, wherein the adjustable flap is integrally formed with the tubular member.

12. The thrombectomy device of claim 1, wherein the side openings are circumferentially arranged around the tubular member.

13. The thrombectomy device of claim 1, wherein the thrombectomy device is configured to engage a thrombus at the treatment site in response to negative pressure.

14. An aspiration catheter comprising:
a proximal end region, a distal end region configured to be positioned at an intravascular treatment site;
a sidewall defining a lumen extending from the proximal end region to the distal end region;
an aspiration opening in the sidewall in the distal end region; and
a cover releasably disposed over the aspiration opening, the cover having a first end coupled to the sidewall and a free second end, the cover adjustable between a closed configuration and an open configuration.

15. A method comprising:
disposing a medical device within a vessel at or adjacent a treatment site, the medical device comprising:
a tubular member, the tubular member having a plurality of side openings in a distal region of the tubular member, and
at least one adjustable flap coupled to the tubular member, wherein the adjustable flap is proximate to a one of the plurality of side openings, and wherein the flap has a first configuration in which the flap at least partially covers the side opening and a second configuration in which the flap is biased away from the side opening; and
transitioning the adjustable flap from the first configuration to the second configuration.

16. The method of claim 15, wherein the adjustable flap is transitioned from the first configuration to the second configuration in response to blood flow contacting a proximal end of the adjustable flap.

17. The method of claim 15, further comprising applying positive pressure to the tubular member, wherein the positive pressure causes the adjustable flap to transition from the first configuration to the second configuration.

18. The method of claim 15, further comprising distally advancing the medical device toward a thrombus at the treatment site, wherein the distal advancement causes the thrombus to abut against a distal end of the adjustable flap, thereby transitioning the adjustable flap from the first configuration to the second configuration.

19. The method of claim 15, further comprising applying negative pressure to the tubular member, thereby causing one or more adjustable flaps to transition from the second configuration to the first configuration.
